(19) United States
(12) Patent Application Publication
(10) Pub. No.: US 2026/0185885 A1
(43) Pub. Date: Jul. 2, 2026

(54) SENSOR, METHOD FOR OPERATING SENSOR, AND WEARABLE DEVICE

(71) Applicant: KOREA ADVANCED INSTITUTE OF SCIENCE AND TECHNOLOGY, Daejeon (KR)

(72) Inventors: Ki-Uk KYUNG, Daejeon (KR); Chongyoung CHUNG, Daejeon (KR); Heeju MUN, Daejeon (KR)

(73) Assignee: KOREA ADVANCED INSTITUTE OF SCIENCE AND TECHNOLOGY, Daejeon (KR)

(21) Appl. No.: 19/233,857

(22) Filed: Jun. 10, 2025

(30) Foreign Application Priority Data
Dec. 27, 2024 (KR) 10-2024-0198440

(51) Int. Cl. G01L 1/24

(52) U.S. Cl. CPC G01L 1/242

(57) ABSTRACT

A sensor according to the present disclosure includes: an optical fiber including a core and a cladding; a light source unit connected to one end of the optical fiber; a detection unit connected to the other end of the optical fiber; a deformation unit spaced apart from the optical fiber; and a body coupled to at least a portion of the optical fiber. The light source unit provides light to the optical fiber, and the detection unit detects light transmitted through the optical fiber and generates light quantity data. The deformation unit has a contact surface with the optical fiber in response to external pressure and has a refractive index greater than that of the cladding. The optical fiber includes a curved portion having a curvature smaller than a critical escape curvature. The detection unit compares contact light quantity data.

This application claims priority to and benefits of Korean Patent Application No. 10-2024-0198440 under 35 U.S. C § 119, filed on Dec. 27, 2024, in the Korean Intellectual Property Office, the contents of which are incorporated herein in its entirety by reference.

### BACKGROUND

### 1. Field

The present disclosure relates to a sensor, a method for operating the sensor, and a wearable device. More particularly, the present disclosure relates to a sensor, a method for operating the sensor, and a wearable device capable of optical sensing without electromagnetic interference.

### 2. Description of the Related Art

In recent years, technologies capable of accurately measuring pressure with high sensitivity have been developed across a variety of fields such as artificial skin, displays, biometric monitoring, and wearable sensors. Particularly, there has been growing interest in research directed toward biometric monitoring using wearable or garment-integrated pressure sensors and in recognizing user intent through Force Myography (FMG).

Wearable pressure sensors typically require the capability to detect very small pressure ranges, necessitating high sensitivity within narrow ranges. Furthermore, flexible sensor technologies that can maintain consistent performance regardless of external conditions such as skin environments involving hair or perspiration are increasingly important.

Accordingly, the present disclosure addresses the development of a sensor that can be directly embedded into fabrics or the like, is thin and flexible, and exhibits high sensitivity within low-force ranges.

### SUMMARY

The present disclosure is directed to providing a sensor, a method for operating the sensor, and a wearable device. The sensor includes an optical fiber having a curved portion with a predetermined curvature and is configured to measure an external pressure based on a light loss caused by contact with the curved portion in response to the applied pressure.

According to an embodiment of the present disclosure, a sensor may include a first optical fiber, a first light source unit, a first detection unit, a deformation unit, and a body.

The first optical fiber may include a core and a cladding.

The first light source unit may be connected to one end of the first optical fiber and may be configured to provide light to the first optical fiber.

The first detection unit may be connected to the other end of the first optical fiber and may be configured to detect light transmitted through the first optical fiber and generate light quantity data.

The deformation unit may be spaced apart from the first optical fiber and may have a first contact surface that comes into contact with the first optical fiber in response to a first external pressure.

The body may be coupled to at least a portion of the first optical fiber.

The first optical fiber may include a first curved portion having a first curvature.

The first curved portion may be disposed between the body and the deformation unit and may contact the first contact surface in response to the first external pressure.

The deformation unit may have a refractive index greater than that of the cladding.

The first curvature may be smaller than the critical escape curvature of the first optical fiber.

The first detection unit may be configured to compare first contact light quantity data, obtained when the deformation unit contacts the first optical fiber, with preset reference data, and calculate the magnitude of the pressure applied to the first contact surface from outside based on a light loss ratio.

According to an embodiment, the sensor may further include a spacer disposed between the deformation unit and the body.

In an embodiment, the first curved portion may have a convex shape oriented toward the deformation unit.

In an embodiment, the deformation unit may further include a second contact surface that contacts the first optical fiber in response to a second external pressure.

The first optical fiber may further include a second curved portion spaced apart from the first curved portion.

The second curved portion may contact the second contact surface in response to the second external pressure.

In an embodiment, the second curved portion may be disposed between the body and the deformation unit and may have a convex shape oriented toward the deformation unit.

In an embodiment, the second curved portion may have a second curvature, which may be smaller than the first curvature.

In an embodiment, a first distance between the first curved portion and the deformation unit may be substantially equal to a second distance between the second curved portion and the deformation unit.

The first optical fiber may further include a lower curved portion connected to the first curved portion and the second curved portion.

The lower curved portion may be exposed from the body in a direction opposite to the deformation unit and may have a concave shape.

In an embodiment, the space between the deformation unit and the body may be filled with air.

The body may be formed of a flexible material.

The first optical fiber may be woven into the body.

In an embodiment, the first optical fiber may have an integral shape.

The sensor may further include a second optical fiber, a second light source unit, and a second detection unit.

The second optical fiber may be spaced apart from the first optical fiber, coupled to the body, and may include a core and a cladding.

The second light source unit may be connected to one end of the second optical fiber and may be configured to provide light to the second optical fiber.

The second detection unit may be connected to the other end of the second optical fiber and may be configured to detect light transmitted through the second optical fiber and generate light quantity data.

The deformation unit may have a second contact surface that comes into contact with the second optical fiber in response to a second external pressure applied at a location different from the first external pressure.

The second optical fiber may include a second curved portion having the first curvature.

The second curved portion may be disposed between the body and the deformation unit and may contact the second contact surface of the deformation unit in response to the second external pressure.

The second detection unit may be configured to compare second contact light quantity data, obtained when the deformation unit contacts the second optical fiber, with preset reference data, and calculate the magnitude of the pressure applied to the first contact surface from outside based on the light loss ratio.

In an embodiment, the first optical fiber and the second optical fiber may extend in parallel along a first direction.

The first optical fiber and the second optical fiber may be spaced apart from each other along a second direction intersecting the first direction.

In an embodiment, the first optical fiber may extend along the first direction, and the second optical fiber may extend along the second direction intersecting the first direction.

In an embodiment, the body may include: a ring-shaped spacer; and a rod-shaped supporter extending from an inner surface of the spacer and coupled to at least a portion of the first optical fiber.

The first optical fiber may include a coupling portion that is wound at least once around the supporter.

The coupling portion may include the first curved portion.

In an embodiment, the supporter may include: a main supporter extending in a first direction; and at least two branch supporters extending from the main supporter in at least two different directions intersecting the first direction.

The coupling portion may be wound at least once around the main supporter and at least once around each of the branch supporters.

According to an embodiment of the present disclosure, a method for operating a sensor may be provided. The sensor may include an optical fiber, a deformation unit, a light source unit, a detection unit, and a body.

The optical fiber may include a curved portion having a curvature smaller than a critical escape curvature.

The deformation unit may be spaced apart from the optical fiber and may have a contact surface contacting with at least a portion of the optical fiber in response to an external pressure.

The light source unit may be configured to provide light to the optical fiber.

The detection unit may be configured to detect light transmitted through the optical fiber and to calculate the magnitude of the external pressure applied to the sensor.

The body may be coupled to at least a portion of the optical fiber.

The method for operating the sensor may include a first step and a second step.

In the first step, the curved portion of the optical fiber may come into contact with the deformation unit due to pressure applied from outside.

In the first step, the detection unit may generate contact light quantity data from the light transmitted through the optical fiber.

In the second step, the detection unit may compare the contact light quantity data with preset reference data and calculate pressure data based on a light loss ratio.

In an embodiment, the sensor may be coupled to a garment at a position corresponding to a heart of a user wearing the garment. The first step and the second step may be performed repeatedly, and the detection unit may output the pressure data in real time to derive a pulse rate.

In another embodiment, the sensor may be coupled to a wristwatch at a position corresponding to the wrist of a user wearing the wristwatch. The first step and the second step may be performed repeatedly, and the detection unit may output the pressure data in real time to derive a pulse rate.

According to an embodiment of the present disclosure, a wearable device may include a piece of equipment worn by a user, a plurality of sensors, and a communication module.

Each of the plurality of sensors may be coupled to the equipment and may be configured to detect pressure applied from outside.

The communication module may be configured to provide location information of the equipment where the pressure is detected and information regarding the magnitude of the pressure.

The sensors may each include an optical fiber, a light source unit, a detection unit, a deformation unit, and a body.

The optical fiber may include a core and a cladding.

The light source unit may be connected to one end of the optical fiber and may be configured to provide light to the optical fiber.

The detection unit may be connected to the other end of the optical fiber and may be configured to detect light transmitted through the optical fiber and generate light quantity data.

The deformation unit may be spaced apart from the optical fiber and may have a contact surface contacting with the optical fiber in response to external pressure.

The body may be coupled to at least a portion of the optical fiber.

The optical fiber may include a curved portion having a predetermined curvature.

The curved portion may be disposed between the body and the deformation unit and may come into contact with the contact surface in response to the external pressure.

The deformation unit may have a refractive index greater than that of the cladding.

The predetermined curvature may be smaller than the critical escape curvature of the optical fiber.

The detection unit may be configured to compare contact light quantity data, obtained when the deformation unit contacts the optical fiber, with preset reference data and calculate the magnitude of the pressure applied to the contact surface from outside based on a light loss ratio.

According to an embodiment of the present disclosure, the sensor, the method for operating the sensor, and the wearable device are capable of measuring external pressure without direct deformation of the optical fiber, are free of electromagnetic interference, and can be integrated with thin and flexible materials. As a result, the sensor, method of operation, and wearable device provide improved reliability and precision.

### BRIEF DESCRIPTION OF THE DRAWINGS

These and/or other features will become apparent and more readily appreciated from the following description of the embodiments, taken in conjunction with the accompanying drawings in which:

FIG. 1 is a schematic diagram illustrating a sensor according to an embodiment of the present disclosure;

FIG. 2 is a plan view illustrating a sensor according to an embodiment of the present disclosure;

FIG. 3A is a cross-sectional view taken along line AA′ of FIG. 2;

FIG. 3B is an enlarged view of region BB1 in FIG. 3A;

FIG. 4A is a cross-sectional view illustrating a sensor according to an embodiment;

FIG. 4B is an enlarged view of region BB2 in FIG. 3B;

FIG. 5 is a schematic diagram illustrating a sensor according to an embodiment;

FIG. 6A is a perspective view illustrating a sensor according to an embodiment;

FIG. 6B is another perspective view illustrating a sensor according to an embodiment;

FIG. 6C is a plan view illustrating a sensor according to an embodiment;

FIG. 6D is a cross-sectional view taken along line CC′ of FIG. 6C;

FIG. 6E is a cross-sectional view illustrating a sensor according to an embodiment;

FIG. 7 is a perspective view showing a partially disassembled sensor according to an embodiment;

FIG. 8 is another perspective view showing a partially disassembled sensor according to an embodiment;

FIG. 9 is a front view illustrating a sensor according to an embodiment;

FIG. 10 is a perspective view illustrating a sensor according to an embodiment; and

FIG. 11 is a plan view illustrating a sensor according to an embodiment.

### DETAILED DESCRIPTION

It is to be understood that the terms such as “include” or “comprise,” and variations thereof, as used in the specification, are intended to indicate the presence of the stated features, numbers, steps, operations, elements, parts, or combinations thereof, but do not preclude the presence or addition of one or more other features, numbers, steps, operations, elements, parts, or combinations thereof.

Further, when an element is described as being “on” another element, this includes being located above or below the other element, and does not necessarily imply being located above in the direction of gravity.

In addition, when it is stated that one element is “connected” or “coupled” to another element, such connection or coupling may be either direct or indirect through another element.

Also, the terms “first,” “second,” etc., used in describing certain elements are only used to distinguish one element from another and do not imply any limitation on the nature, order, or priority of the elements.

The terminology used in the present specification and claims should not be interpreted as being limited to common or dictionary meanings, and should be interpreted in accordance with the spirit of the disclosure based on the principle that the inventor is entitled to define the terms as needed to best explain the disclosure.

Furthermore, singular expressions used in the present application are intended to include plural forms unless the context clearly dictates otherwise.

Hereinafter, preferred embodiments of the present disclosure will be described in more detail with reference to the accompanying drawings. In the drawings, the proportions and dimensions of the elements may be exaggerated for purposes of illustration and explanation of the technical content.

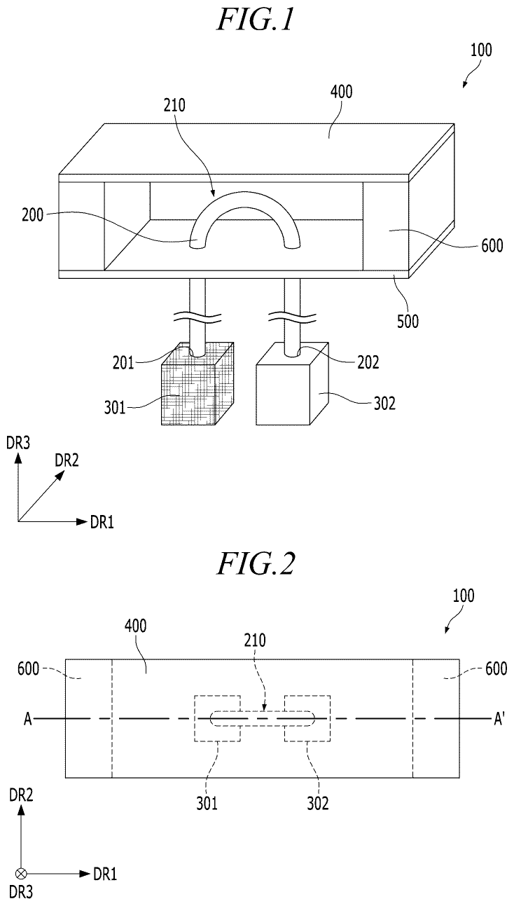

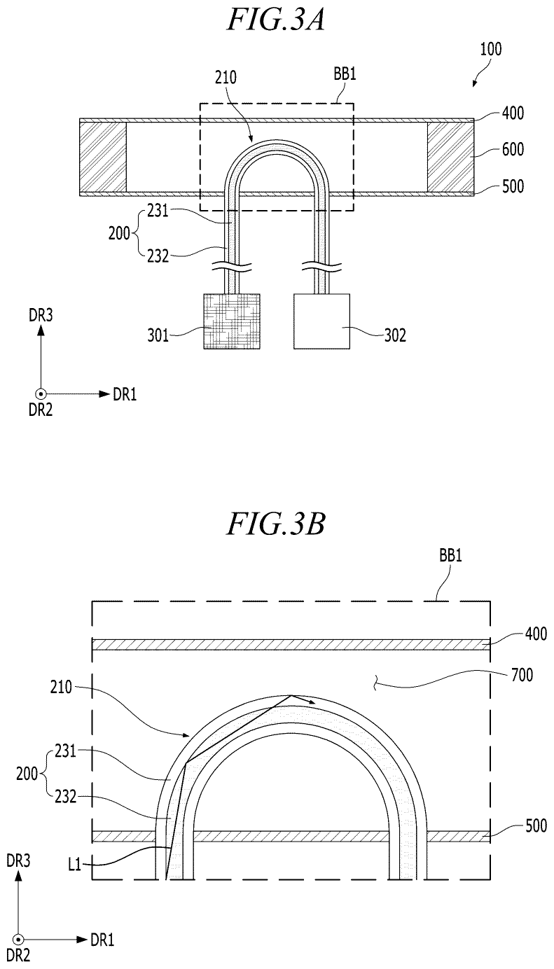

FIG. 1 is a schematic diagram illustrating a sensor according to an embodiment of the present disclosure. FIG. 2 is a plan view illustrating the sensor according to an embodiment of the present disclosure.

Referring to FIGS. 1 and 2, a sensor 100 according to an embodiment may include an optical fiber 200, a light source unit 301, a detection unit 302, a deformation unit 400, a body 500, and a spacer 600.

One end 201 of the optical fiber 200 may be connected to the light source unit 301, and the other end 202 may be connected to the detection unit 302. The light source unit 301 may be configured to provide light to the optical fiber 200, and the detection unit 302 may be configured to measure the amount of light transmitted through the optical fiber 200.

The deformation unit 400 may be disposed spaced apart from the optical fiber 200. In an embodiment, the deformation unit 400 may extend parallel to a plane defined by a first direction DR1 and a second direction DR2.

In an embodiment, the deformation unit 400 may be made of a polymer material.

In an embodiment, the deformation unit 400 may be subject to tensile force in all four directions and may be coupled to the spacer 600, as will be described later. Therefore, when no external pressure is applied to the sensor 100, sagging of the deformation unit 400 may be prevented.

When pressure is applied from outside, temporary deformation of the deformation unit 400 may occur at the location of the applied pressure, allowing the deformation unit 400 to contact the optical fiber 200. Further details regarding this interaction will be provided later.

The body 500 may serve as a base member to which the optical fiber 200 is coupled.

In an embodiment, the body 500 may be formed of a flexible material, and the optical fiber 200 may be woven into the body 500. However, the embodiment is not limited thereto; in another embodiment, the body 500 may be made of a rigid material, and the optical fiber 200 may be wound and coupled to a portion of the body 500. This will also be described in more detail later.

The spacer 600 may be disposed between the deformation unit 400 and the body 500 and may serve to maintain a separation between the optical fiber 200 and the deformation unit 400. Additionally, the spacer 600 may serve to maintain the structural shape of the sensor 100.

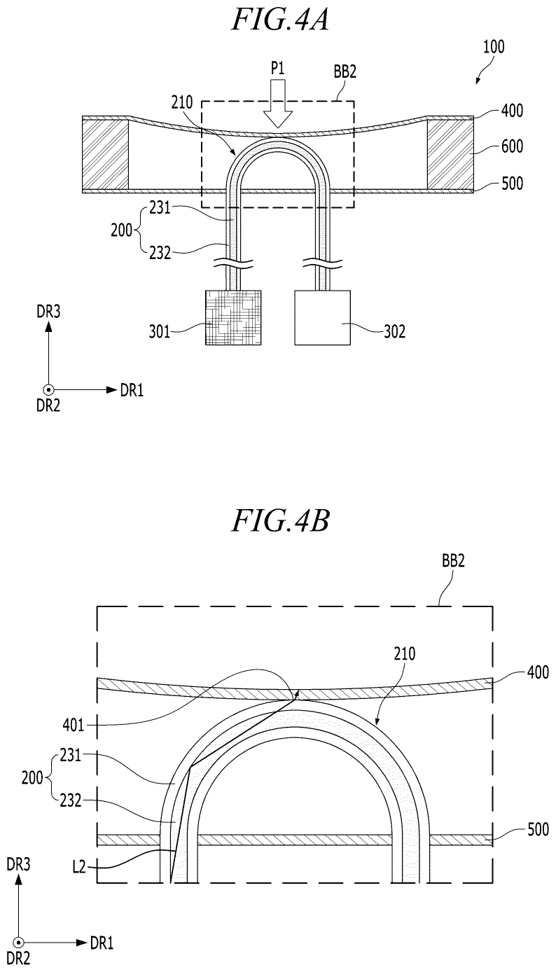

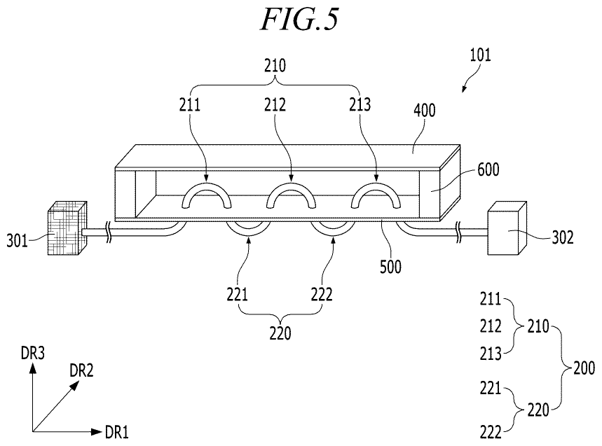

FIG. 3A is a cross-sectional view taken along line AA′ of FIG. 2. FIG. 3B is an enlarged view of region BB1 in FIG. 3A. FIG. 4A is a cross-sectional view illustrating a sensor according to an embodiment. FIG. 4B is an enlarged view of region BB2 in FIG. 3B.

FIGS. 3A and 3B illustrate a portion of the sensor 100 in a state where no external pressure is applied.

Referring to FIGS. 3A and 3B, the optical fiber 200 according to an embodiment of the present disclosure may include a core 231 and a cladding 232. The core 231 and the cladding 232 may provide a passage through which light provided from the light source unit 301 travels. The cladding 232 may be made of a material having a lower refractive index than the core 231 and may surround the core 231. Light may be totally internally reflected within the core 231. Some of the light may not be totally internally reflected and may enter the cladding 232, and a portion of this light may be reflected at a boundary between the cladding 232 and the external environment and return into the interior of the optical fiber 200.

In an embodiment of the present disclosure, the optical fiber 200 may include a curved portion 210 disposed between the deformation unit 400 and the body 500. The curved portion 210 may have a predetermined curvature smaller than a critical escape curvature.

As used herein, the term “critical escape curvature” refers to the minimum curvature at which at least a portion of light fails to be totally internally reflected within the optical fiber 200 and escapes to the outside. In a curved portion 210 having a curvature smaller than the critical escape curvature, even if light is incident into the cladding 232 due to a lack of total internal reflection in the core 231, the light may not escape into an external space 700. The external space 700 refers to the space outside the optical fiber 200, specifically the space between the deformation unit 400 and the body 500.

Referring again to FIGS. 3A and 3B, in the state where no external pressure is applied to the sensor 100, light L1 traveling through the optical fiber 200 may remain within the fiber and not escape into the surrounding environment.

In an embodiment, the external space 700 may be filled with air.

In an embodiment, the curved portion 210 may have a convex shape oriented toward the deformation unit 400.

Because the air filled in the external space 700 has a lower refractive index than the cladding 232, light within the curved portion 210, of which the curvature is less than the critical escape curvature, may be totally internally reflected at the boundary of the cladding 232.

FIGS. 4A and 4B illustrate a portion of the sensor 100 in a state where external pressure is applied.

Referring to FIGS. 4A and 4B, when external pressure P1 is applied to the deformation unit 400, temporary deformation may occur in the deformation unit 400, causing the deformation unit 400 to come into contact with the optical fiber 200. In such a case, the deformation unit 400 may have a contact surface 401 that contacts the curved portion 210. The area of the contact surface 401 may be proportional to the magnitude of the external pressure P1.

In an embodiment, the external pressure P1 may be of a magnitude such that the predetermined curvature of the curved portion 210 remains substantially unchanged. Upon contact between the deformation unit 400 and the curved portion 210, a portion of light L2 traveling through the optical fiber 200 may escape through the contact surface 401 into the deformation unit 400. Since the deformation unit 400 is made of a material having a refractive index higher than that of the cladding 232, the portion of light L2 may not be totally internally reflected at the contact surface 401 and may instead escape to outside through the deformation unit 400.

Referring to FIGS. 3A through 4B, in an embodiment, at the sensor 100 in the absence of applied pressure (FIG. 3A), the light L1 provided from the light source unit 301 (FIG. 3B) may reach the detection unit 302 without escaping into the space 700 outside the optical fiber 200. In contrast at the sensor 100 in the presence of external pressure P1 (FIG. 4A), the portion of light L2 emitted from the light source unit 301 (FIG. 4B) may escape into the space 700 outside the optical fiber 200, and the remaining light that has not escaped may reach the detection unit 302.

The detection unit 302 may, in the absence of external pressure, detect the light emitted from the light source unit 301 and transmitted through the optical fiber 200 and may store it as preset reference data. In the state where the external pressure P1 is applied to the deformation unit 400 (FIG. 4B), the detection unit 302 may detect the remaining light that has not been lost through the deformation unit 400 and generate first contact light quantity data.

In an embodiment of the present disclosure, the detection unit 302 may be configured to compare the preset reference data and the first contact light quantity data and measure and calculate the magnitude of the pressure P1 (FIG. 3B) applied to the contact surface 401 (FIG. 3B) from outside based on a light loss ratio. The external pressure P1 (FIG. 3B) applied to the sensor 100 may be minute pressure that does not cause direct deformation of the optical fiber 200. The sensor 100 according to an embodiment of the present disclosure may be configured to detect minute pressures. The sensor 100 may be configured to use light to detect the external pressure P1 (FIG. 3B) and may be capable of precise sensing that is substantially free from electromagnetic interference. Additionally, in an embodiment, the optical fiber 200 may be directly woven into a flexible material, facilitating easy and versatile sensor fabrication. In another embodiment, the optical fiber 200 may not be directly deformed, exhibiting improved reliability.

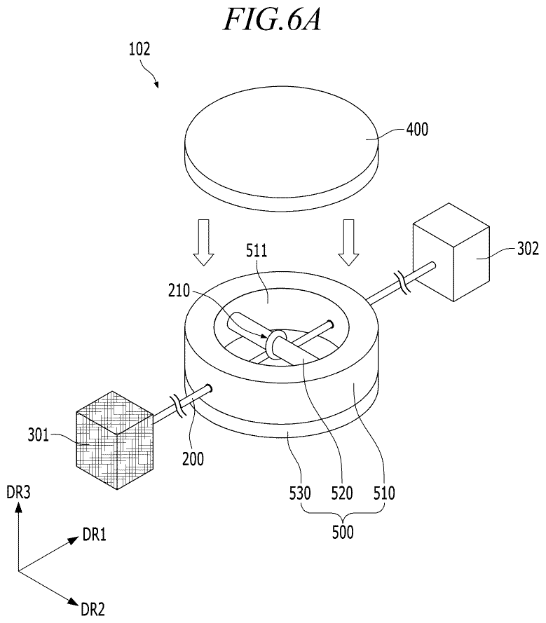

FIG. 5 is a schematic diagram illustrating a sensor according to an embodiment. Referring to FIG. 5, the optical fiber 200 included in a sensor 101 according to an embodiment may include an upper curved portion 210 and a lower curved portion 220. The upper curved portion 210 may include first to third upper curved portions 211, 212, 213. The first to third upper curved portions 211, 212, 213 may be disposed between the deformation unit 400 and the body 500, may be spaced apart from one another along the first direction DR1, and may have a convex shape oriented toward the deformation unit 400.

In an embodiment of the disclosure, each of the first to third upper curved portions 211, 212, 213 may have a curvature smaller than the critical escape curvature.

In an embodiment, the first upper curved portion 211, the second upper curved portion 212, and the third upper curved portion 213 may each have substantially the same curvature. However, the embodiment is not limited thereto, and the first to third upper curved portions 211, 212, 213 may have different curvatures. Further details will be described later.

The lower curved portion 220 may be exposed from the body 500 in a direction opposite to the deformation unit 400 and may have a concave shape. The lower curved portion 220 may include a first lower curved portion 221 and a second lower curved portion 222. The first lower curved portion 221 may connect the first upper curved portion 211 and the second upper curved portion 212, and the second lower curved portion 222 may connect the second upper curved portion 212 and the third upper curved portion 213.

The sensor 101 according to an embodiment may include the first to third upper curved portions 211, 212, 213, which are spaced apart from one another along the first direction DR1. When the deformation unit 400 contacts any one of the first to third upper curved portions 211, 212, 213 due to external pressure applied to the sensor 101, the detection unit 302 may detect the applied external pressure.

In an embodiment, the number of upper curved portions included in the sensor may be proportional to the range of external pressures that the sensor can detect.

In an embodiment, the optical fiber 200 may have an integral shape.

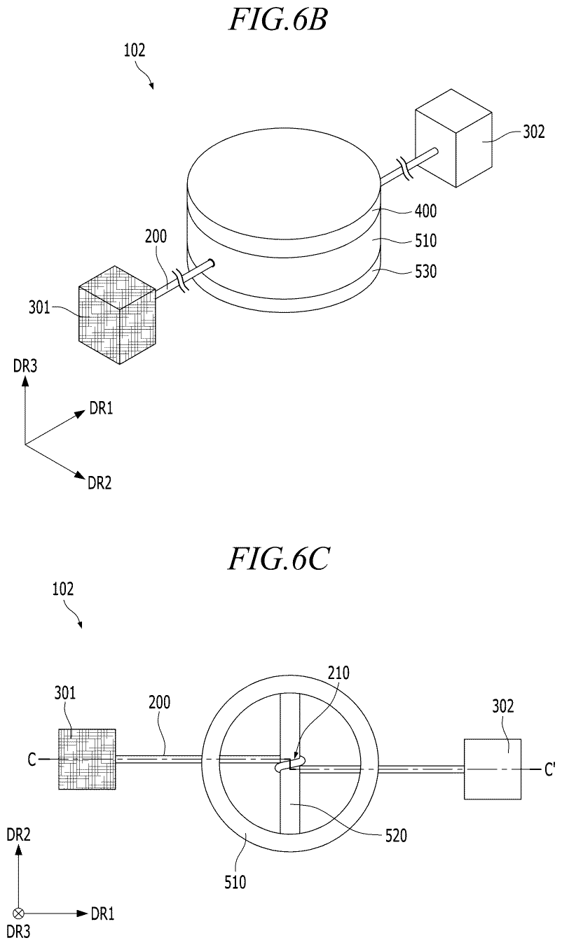

FIG. 6A is a perspective view illustrating a sensor according to an embodiment. FIG. 6B is another perspective view illustrating a sensor according to an embodiment. FIG. 6C is a plan view illustrating a sensor according to an embodiment. FIG. 6D is a cross-sectional view taken along line CC′ of FIG. 6C. FIG. 6E is a cross-sectional view illustrating a sensor according to an embodiment.

In FIG. 6A, some components are shown separated for the purpose of easier understanding.

Referring to FIGS. 6A through 6D, a sensor 102 according to an embodiment may include an optical fiber 200, a light source unit 301, a detection unit 302, a deformation unit 400, and a body 500.

In an embodiment, the body 500 may include a ring-shaped spacer 510 and a supporter 520.

The ring-shaped spacer 510 may include an inner surface 511 from which the supporter 520 extends. The ring-shaped spacer 510 may maintain a separation between the deformation unit 400 and the optical fiber 200 including its curved portion 210 in the absence of external pressure, thereby preventing contact between them.

In an embodiment, the body 500 may further include a bottom portion 530. The bottom portion 530 may be coupled to the ring-shaped spacer 510. However, the embodiment is not limited thereto, and, in other embodiments, multiple sensors may be provided and may be simultaneously coupled to a single bottom portion. In an embodiment, the bottom portion may be an article of clothing or equipment worn by a user, and the sensors may be coupled partially or entirely to the clothing or equipment.

In an embodiment, the optical fiber 200 may pass through one side of the ring-shaped spacer 510, extend into the ring-shaped spacer 510, and exit through the opposite side to the exterior of the ring-shaped spacer 510. One end of the optical fiber 200 may be connected to the light source unit 301, which is configured to provide light, and the other end may be connected to the detection unit 302, which is configured to detect the transmitted light. The optical fiber 200 may include a coupling portion 240 that is wound around the supporter 520 inside the ring-shaped spacer 510.

In an embodiment, the coupling portion 240 may be wound around the supporter 520 at least once. The coupling portion 240 may include a curved portion 210 having a convex shape oriented toward the deformation unit 400 and a predetermined curvature that may be smaller than the critical escape curvature.

In a state where no external pressure is applied, light emitted from the light source unit 301 may pass through the optical fiber 200 and be detected by the detection unit 302.

Referring to FIG. 6E, when pressure P2 is applied to the sensor 102 from outside, temporary deformation of the deformation unit 400 may occur, resulting in contact between the deformation unit 400 and the curved portion 210. At this time, the deformation unit 400 may include a contact surface 401 that contacts the curved portion 210. A portion of the light provided by the light source unit 301 may escape from the optical fiber 200 through the deformation unit 400, and the detection unit 302 may measure the light transmitted through the optical fiber 200 and calculate the magnitude of the pressure P2 applied from outside.

The sensor 102 illustrated in FIGS. 6A through 6E may be readily fabricated as an independent modular sensor having an ultra-compact size, which can be conveniently attached to a specific location on equipment worn by a user. For example, the user may attach the sensor 102 according to an embodiment to a localized area where pressure measurement is desired, enabling precise measurement of the pressure applied to that specific location.

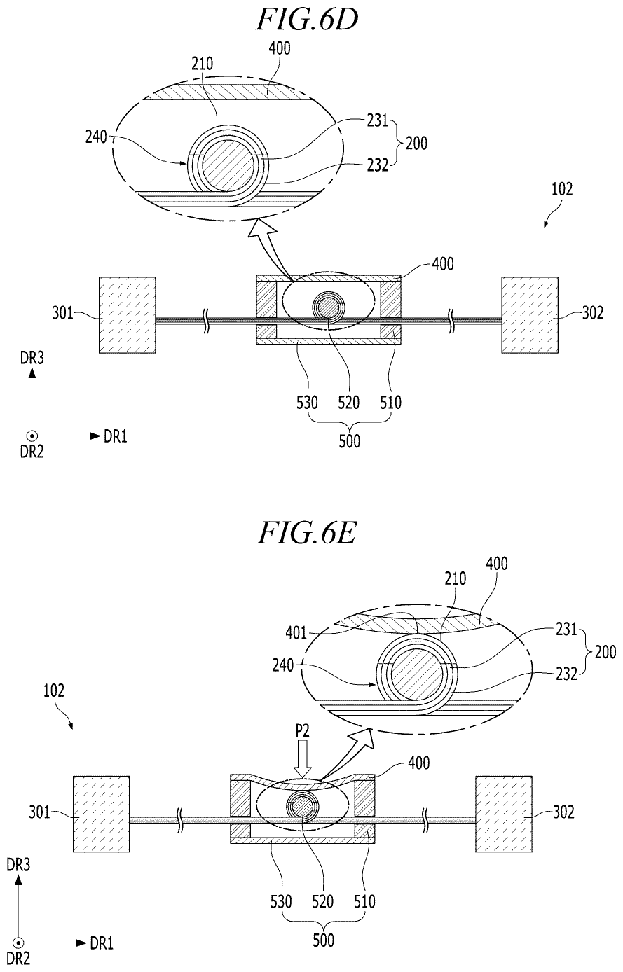

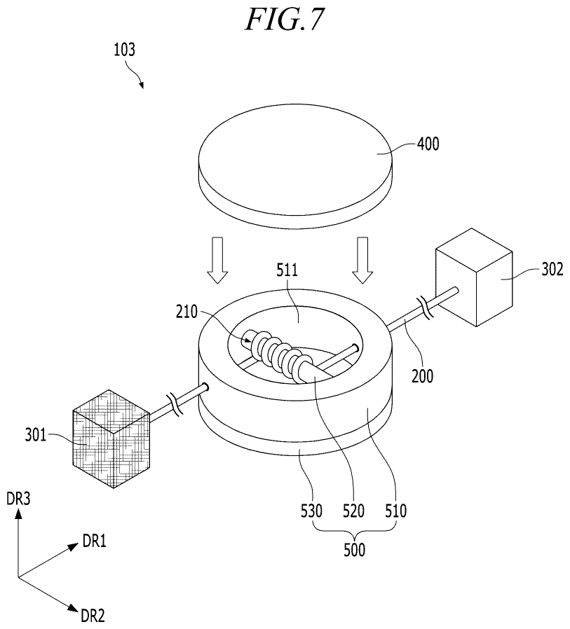

FIG. 7 is a perspective view showing a partially disassembled sensor according to an embodiment. FIG. 8 is another perspective view showing a partially disassembled sensor according to an embodiment.

Referring to FIG. 7, in a sensor 103 according to an embodiment, the optical fiber 200 may be wound around the supporter 520 at least once inside the body 500. While the optical fiber 200 is depicted in FIG. 7 to be wound around the supporter 520 five times, the embodiment is not limited thereto, and the number of windings of the optical fiber 200 around the supporter 520 may be appropriately adjusted.

The optical fiber 200 may include a number of curved portions 210 proportional to the number of times it is wound around the supporter. When external pressure is applied, the deformation unit 400 may contact the optical fiber 200 over a larger contact area, thereby enhancing the precision of the sensor 103.

Referring to FIG. 8, a sensor 104 according to an embodiment may include a supporter 520 including a main supporter 521, a first branch supporter 522, and a second branch supporter 523. The main supporter 521 may extend in the first direction DR1 from the inner surface 511 of the spacer 510, and the first branch supporter 522 and the second branch supporter 523 may extend in different directions intersecting the first direction DR1, respectively. The optical fiber 200 may be wound at least once around each of the main supporter 521, the first branch supporter 522, and the second branch supporter 523 and may include curved portions 210 on each of the main supporter 521, the first branch supporter 522, and the second branch supporter 523. By including curved portions 210 on the supporters extending in different directions, the optical fiber 200 may have a uniform contact surface with the deformation unit 400, regardless of the direction from which external pressure is applied, thereby improving the reliability of the sensor 104. The embodiment is not limited to this configuration, and the number of branch supporters extending from the main supporter may be at least two or more.

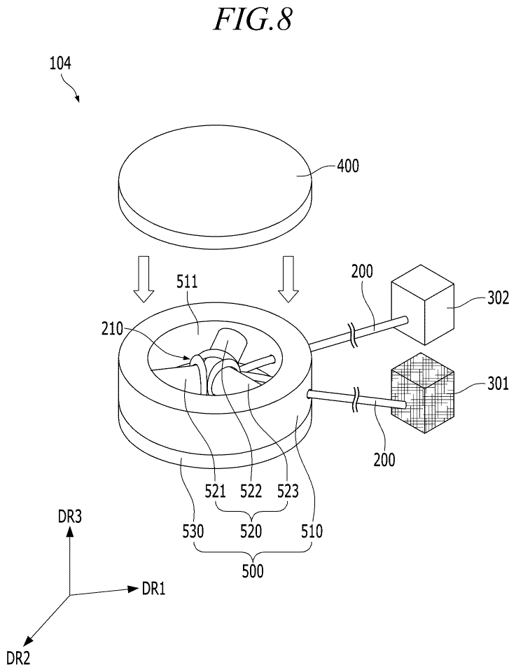

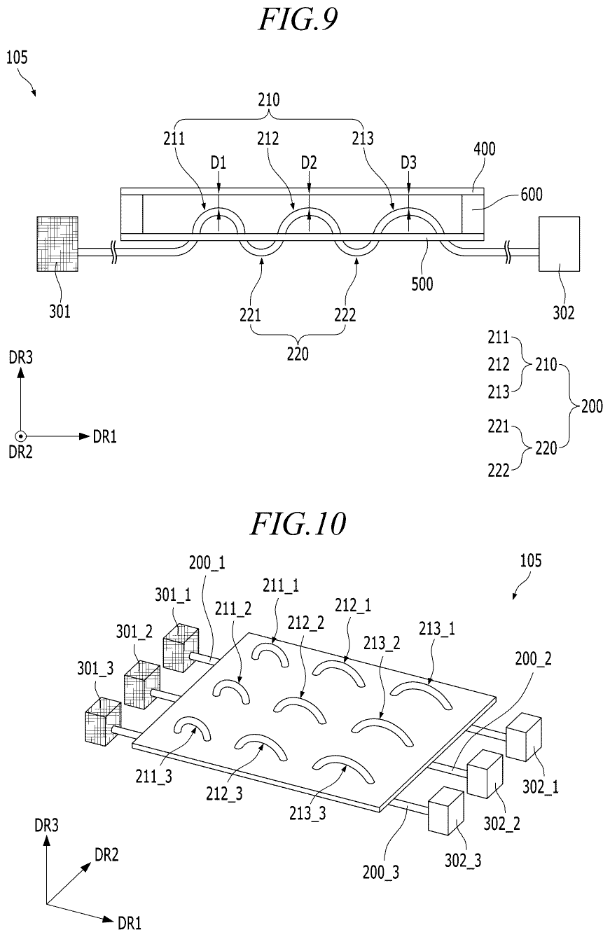

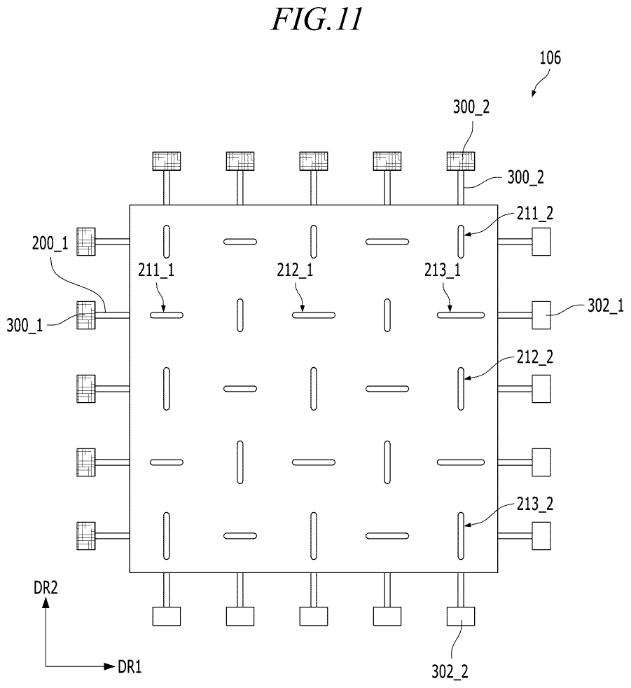

FIG. 9 is a front view illustrating a sensor according to an embodiment. FIG. 10 is a perspective view illustrating a sensor according to an embodiment. FIG. 11 is a plan view illustrating a sensor according to an embodiment. In FIGS. 9 to 11, certain components are omitted for the purpose of easier understanding.

Referring to FIG. 9, in a sensor 105 according to an embodiment, the curved portion 210 may include a first curved portion 211, a second curved portion 212, and a third curved portion 213.

The first curved portion 211 may have a curvature smaller than the critical escape curvature. The second curved portion 212 may have a curvature smaller than that of the first curved portion 211. The third curved portion 213 may have a curvature smaller than that of the second curved portion 212.

A first distance D1 between the first curved portion 211 and the deformation unit 400 may be substantially equal to a second distance D2 between the second curved portion 212 and the deformation unit 400 and a third distance D3 between the third curved portion 213 and the deformation unit 400.

The first to third curved portions 211, 212, 213 may each have a curvature smaller than the critical escape curvature and may have different curvatures. When the same magnitude of external pressure is applied, causing contact between each of the first to third curved portions 211, 212, 213 and the deformation unit 400, the contact area between each of the first to third curved portions 211, 212, 213 and the deformation unit 400 may differ. Moreover, since the first to third curved portions 211, 212, 213 have different curvatures, the angle of light incident at the contact surfaces may also differ, and accordingly, the amount of light escaping through the deformation unit 400 may vary.

When a same magnitude of external pressure is applied such that each of the first to third curved portions 211, 212, 213 has a contact surface with the deformation unit 400, the sensor 105 having these characteristics may have different widths of variation in the amount of light detected by the detection unit 302. Through this, the detection unit 302 may infer which of the curved portions 211, 212, 213 was in contact with the deformation unit 400 due to the applied external pressure, thereby enabling more detailed acquisition of the position information at which the external pressure was applied.

Referring to FIG. 10, the sensor 105 according to an embodiment may include first to third optical fibers 200_1, 200_2, 200_3, each extending in the first direction DR1 and spaced apart from one another in the second direction DR2.

One end of the first optical fiber 200_1 may be connected to a first light source unit 301_1, which is configured to provide light, and the other end may be connected to a first detection unit 302_1, which is configured to detect the light transmitted through the first optical fiber 200_1. The first optical fiber 200_1 may include first curved portions 211_1, 212_1, 213_1. These curved portions may have curvatures smaller than the critical escape curvature and may each have a different curvature. The first detection unit 302_1 may be configured to calculate the position and magnitude of external pressure applied along the first direction DR1.

One end of the second optical fiber 200_2 may be connected to a second light source unit 301_2, which is configured to provide light, and the other end may be connected to a second detection unit 302_2, which is configured to detect the light transmitted through the second optical fiber 200_2. The second optical fiber 200_2 may include second curved portions 211_2, 212_2, 213_2, which may have curvatures smaller than the critical escape curvature and different from one another. The second detection unit 302_2 may be configured to calculate the position and magnitude of external pressure applied along the first direction DR1.

One end of the third optical fiber 200_3 may be connected to a third light source unit 301_3, which is configured to provide light, and the other end may be connected to a third detection unit 302_3, which is configured to detect the light transmitted through the third optical fiber 200_3. The third optical fiber 200_3 may include third curved portions 211_3, 212_3, 213_3, which may have curvatures smaller than the critical escape curvature and different from one another. The third detection unit 302_3 may be configured to calculate the position and magnitude of external pressure applied along the first direction DR1.

The sensor 105 according to an embodiment may include optical fibers arranged in rows along the first direction DR1, each having curved portions with different curvatures. In the sensor 105, the optical fibers 200_1, 200_2, 200_3 extending in the first direction DR1 may be arranged in columns extending along the second direction DR2 and spaced apart from one another. Each of the optical fibers 200_1, 200_2, 200_3 may be connected to a corresponding light source unit and detection unit. Accordingly, when external pressure is applied to the sensor 105, the sensor 105 may acquire both the matrix coordinate information of the location where the pressure is applied and the magnitude information of the pressure. The sensor 105 may be incorporated into clothing or equipment to facilitate measuring physical condition information of a user. For example, the sensor 105 according to an embodiment may be attached to a location on clothing corresponding to a user's heart and used to detect heartbeats to calculate a pulse rate. The sensor 106 may also be attached to a location on the equipment corresponding to a user's wrist to detect arterial pulsation and calculate a pulse rate. The equipment may be a wearable item worn on the wrist, including a bracelet, a watch, or an electronic watch, though the type of equipment is not limited to these examples.

Although the sensor 105 according to an embodiment is depicted as having three optical fibers 200_1, 200_2, 200_3, each including three curved portions, the embodiment is not limited thereto.

In an embodiment, the sensor may include at least two or more optical fibers extending in the first direction. One end of each optical fiber may be connected to a different light source unit, and the other end may be connected to a different detection unit. Each optical fiber may include at least two or more curved portions.

Referring to FIG. 11, a sensor 106 may include a plurality of first optical fibers 200_1 extending in the first direction DR1 and a plurality of second optical fibers 200_2 extending in the second direction DR2. The first optical fibers 200_1 may include first curved portions 211_1, 212_1, 213_1 having curvatures smaller than the critical escape curvature and different from one another. The second optical fibers 200_2 may include second curved portions 211_2, 212_2, 213_2 having curvatures smaller than the critical escape curvature and different from one another. In the sensor 106 according to an embodiment, the first optical fibers 200_1 extending in the first direction DR1 and the second optical fibers 200_2 extending in the second direction DR2 may be arranged to intersect with each other. When external pressure is applied, the sensor 106 may identify the detailed location to which the pressure is applied with improved precision.

According to a method for operating the sensor in an embodiment, the sensor may include an optical fiber, a light source unit, a detection unit, a deformation unit, and a body.

One end of the optical fiber may be connected to the light source unit, which is configured to provide light to the optical fiber. The other end of the optical fiber may be connected to the detection unit, which is configured to detect the light transmitted through the optical fiber. The detection unit may be configured to calculate the magnitude of external pressure applied to the sensor based on the detection result. At least a portion of the optical fiber may be coupled to the body. The deformation unit may be disposed spaced apart from the optical fiber and may come into contact with at least a portion of the optical fiber when external pressure is applied.

The optical fiber may include a curved portion having a curvature smaller than the critical escape curvature. The curved portion may be disposed between the body and the deformation unit and may have a convex shape oriented toward the deformation unit.

The method for operating the sensor according to an embodiment may include a first step and a second step.

In the first step, external pressure may cause the deformation unit to come into contact with the curved portion of the optical fiber. At this time, the deformation unit may have a contact surface coming into contact with the optical fiber. Once the deformation unit comes into contact with the optical fiber, a portion of light provided from the light source unit may escape into the deformation unit through the contact surface of the deformation unit. The detection unit may detect the transmitted light and generate contact light quantity data.

In the second step, the detection unit may compare preset reference data with the contact light quantity data and, if the light loss exceeds a threshold value, calculate pressure data based on the loss ratio.

According to the method for operating the sensor, the sensor may calculate the magnitude of external pressure based on the light loss ratio using an optical fiber, thereby avoiding electromagnetic interference and improving reliability. Moreover, the optical fiber includes a curved portion having a defined curvature, and when a contact occurs between the curved portion and the deformation unit in response to minute external pressure, this pressure may be detected with improved precision. Additionally, since the optical fiber does not undergo direct deformation during pressure detection, the sensor may have extended durability, leading to improved reliability.

In the method for operating the sensor according to an embodiment, the sensor may be coupled to a garment at a position corresponding to the user's heart. The first step and the second step may be iteratively performed, and the detection unit may output the pressure data in real time to calculate the user's pulse.

In the method for operating the sensor according to another embodiment, the sensor may be coupled to a wristwatch at a position corresponding to the user's wrist. The first step and the second step may be iteratively performed, and the detection unit may output the pressure data in real time to calculate the user's pulse.

A wearable device according to an embodiment of the present disclosure may include: equipment worn by a user; a plurality of sensors, each coupled to the equipment and configured to detect applied pressure; and a communication module configured to provide location information of the equipment where the pressure is detected and information regarding the magnitude of the detected pressure.

Each of the sensors may include: an optical fiber including a core and a cladding; a light source unit connected to one end of the optical fiber and configured to provide light to the optical fiber; a detection unit connected to the other end of the optical fiber and configured to measure the light transmitted through the optical fiber and generate light quantity data; a deformation unit spaced apart from the optical fiber and having a contact surface that contacts the optical fiber in response to external pressure; and a body coupled to at least a portion of the optical fiber.

The optical fiber may include a curved portion having a predetermined curvature. The curved portion may be disposed between the body and the deformation unit and may come into contact with the contact surface in response to the external pressure. The deformation unit may have a refractive index greater than that of the cladding. The predetermined curvature may be smaller than a critical escape curvature of the optical fiber.

The detection unit may be configured to compare contact light quantity data, obtained when the deformation unit contacts the optical fiber, with preset reference data and calculate the magnitude of pressure applied to the contact surface based on a light loss ratio.

While certain embodiments of the present disclosure have been described herein, anyone ordinarily skilled in the art to which the present disclosure pertains shall appreciate that there may be a variety of modifications and permutations of the present disclosure without departing from the technical ideas and scopes of the present disclosure that are defined in the appended claims. Moreover, it shall be appreciated that the disclosed embodiments are not intended to restrict the present disclosure thereto and that every technical idea within the appended claims and their equivalents is interpreted to be included in the scope of the present disclosure.

What is claimed is:

1. A sensor comprising:
a first optical fiber comprising a core and a cladding;
a first light source unit connected to one end of the first optical fiber and configured to provide light to the first optical fiber;
a first detection unit connected to the other end of the first optical fiber and configured to detect light transmitted through the first optical fiber and generate light quantity data;
a deformation unit spaced apart from the first optical fiber and having a first contact surface configured to contact the first optical fiber in response to a first external pressure; and
a body coupled to at least a portion of the first optical fiber,
wherein the first optical fiber comprises a first curved portion having a first curvature,
wherein the first curved portion is disposed between the body and the deformation unit and configured to contact the first contact surface in response to the first external pressure,
wherein the deformation unit has a refractive index greater than that of the cladding,
wherein the first curvature is smaller than a critical escape curvature of the first optical fiber, and
wherein the first detection unit is configured to compare first contact light quantity data, obtained when the deformation unit contacts the first optical fiber, with preset reference data and calculate a magnitude of pressure applied from outside to the first contact surface based on a light loss ratio.

2. The sensor of claim 1, further comprising a spacer disposed between the deformation unit and the body.

3. The sensor of claim 1, wherein the first curved portion has a convex shape oriented toward the deformation unit.

4. The sensor of claim 1, wherein the deformation unit further comprises a second contact surface configured to contact the first optical fiber in response to a second external pressure,
wherein the first optical fiber further comprises a second curved portion spaced apart from the first curved portion, and
wherein the second curved portion is configured to contact the second contact surface in response to the second external pressure.

5. The sensor of claim 4, wherein the second curved portion is disposed between the body and the deformation unit and has a convex shape oriented toward the deformation unit.

6. The sensor of claim 4, wherein the second curved portion has a second curvature, and wherein the second curvature is smaller than the first curvature.

7. The sensor of claim 6, wherein a first distance between the first curved portion and the deformation unit is substantially equal to a second distance between the second curved portion and the deformation unit.

8. The sensor of claim 4, wherein the first optical fiber further comprises a lower curved portion connected to the first curved portion and the second curved portion, and
wherein the lower curved portion is exposed from the body in a direction opposite to the deformation unit and has a concave shape.

9. The sensor of claim 1, wherein a space between the deformation unit and the body is filled with air.

10. The sensor of claim 1, wherein the body is provided with a flexible material, and
wherein the first optical fiber is woven into the body.

11. The sensor of claim 1, wherein the first optical fiber has an integral shape.

12. The sensor of claim 1, further comprising:
a second optical fiber spaced apart from the first optical fiber, coupled to the body, and comprising a core and a cladding;
a second light source unit connected to one end of the second optical fiber and configured to provide light to the second optical fiber; and
a second detection unit connected to the other end of the second optical fiber and configured to detect light transmitted through the second optical fiber and generate light quantity data,
wherein the deformation unit has a second contact surface configured to contact the second optical fiber in response to a second external pressure applied at a position different from the first external pressure,
wherein the second optical fiber comprises a second curved portion having the first curvature,
wherein the second curved portion is disposed between the body and the deformation unit and configured to contact the second contact surface in response to the second external pressure, and
wherein the second detection unit is configured to compare second contact light quantity data, obtained when the deformation unit contacts the second optical fiber, with preset reference data and calculate a magnitude of pressure applied from outside to the first contact surface based on a light loss ratio.

13. The sensor of claim 12, wherein the first optical fiber and the second optical fiber extend in parallel along a first direction, and
wherein the first optical fiber and the second optical fiber are spaced apart from each other along a second direction intersecting the first direction.

14. The sensor of claim 12, wherein the first optical fiber extends along a first direction, and
wherein the second optical fiber extends along a second direction intersecting the first direction.

15. The sensor of claim 1, wherein the body comprises:
a ring-shaped spacer; and
a rod-shaped supporter extending from an inner surface of the spacer and coupled to at least a portion of the first optical fiber,
wherein the first optical fiber comprises a coupling portion wound at least once around the supporter, and
wherein the coupling portion comprises the first curved portion.

16. The sensor of claim 1, wherein the supporter comprises:
a main supporter extending in a first direction; and
at least two or more branch supporters extending from the main supporter in two or more different directions intersecting the first direction,
wherein the coupling portion is wound at least once around the main supporter, and
wherein the coupling portion is wound at least once around each of the branch supporters.

17. A method for operating a sensor comprising an optical fiber including a curved portion having a curvature smaller than a critical escape curvature, a deformation unit spaced apart from the optical fiber and having a contact surface with at least a portion of the optical fiber in response to external pressure, a light source unit configured to provide light to the optical fiber, a detection unit configured to detect light transmitted through the optical fiber and calculate a magnitude of external pressure applied to the sensor, and a body coupled to at least a portion of the optical fiber, the method comprising:
a first step of contacting the curved portion of the optical fiber with the deformation unit in response to pressure applied from outside, wherein the detection unit generates contact light quantity data from the light transmitted through the optical fiber; and
a second step of comparing the contact light quantity data with preset reference data at the detection unit and calculating pressure data based on a light loss ratio.

18. The method of claim 17, wherein the sensor is coupled to a garment at a position corresponding to a heart of a user wearing the garment,
wherein the first step and the second step are iteratively performed, and
wherein the detection unit outputs the pressure data in real time to calculate a pulse.

19. The method of claim 17, wherein the sensor is coupled to a wristwatch at a position corresponding to a wrist of a user wearing the wristwatch,
wherein the first step and the second step are iteratively performed, and
wherein the detection unit outputs the pressure data in real time to calculate a pulse.

20. A wearable device comprising:
equipment worn by a user;
a plurality of sensors, each being coupled to the equipment and configured to detect pressure applied from outside; and
a communication module configured to provide location information of the equipment where the pressure is detected and information regarding the magnitude of the detected pressure,
wherein each of the sensors comprises:
an optical fiber comprising a core and a cladding;
a light source unit connected to one end of the optical fiber and configured to provide light to the optical fiber;
a detection unit connected to the other end of the optical fiber and configured to detect light transmitted through the optical fiber and generate light quantity data;
a deformation unit spaced apart from the optical fiber and having a contact surface configured to contact the optical fiber in response to external pressure; and
a body coupled to at least a portion of the optical fiber,
wherein the optical fiber comprises a curved portion having a predetermined curvature,
wherein the curved portion is disposed between the body and the deformation unit and configured to contact the contact surface in response to the external pressure,
wherein the deformation unit has a refractive index greater than that of the cladding,
wherein the predetermined curvature is smaller than a critical escape curvature of the optical fiber, and
wherein the detection unit is configured to compare contact light quantity data, obtained when the deformation unit contacts the optical fiber, with preset reference data and calculate a magnitude of pressure applied to the contact surface from outside based on a light loss ratio.
